(19) United States
(12) Patent Application Publication
(10) Pub. No.: US 2006/0269443 A1
(43) Pub. Date: Nov. 30, 2006

(54) Method and apparatus for treating stored crops utilizing recycled air

(76) Inventors: William A. Keim, Fort Wayne, IN (US); Michael J. Keim, Barberton, OH (US); John R. Keim, Barberton, OH (US)

Correspondence Address: SAND & SEBOLT, AEGIS TOWER, SUITE 1100, 4940 MUNSON STREET, NW, CANTON, OH, 44718-3615, US

(21) Appl. No.: 11/135,714

(22) Filed: May 24, 2005

(51) Int. Cl. A01N 25/00; A61L 2/00

(52) U.S. Cl. 422/38

(57) ABSTRACT

A stored crop treatment system includes a crop storage chamber and a gas stream circulation pathway which includes a re-circulation loop comprising the crop storage chamber. A feed mechanism feeds stored crop treatment material into a stream of gas moving through the re-circulation loop wherein the stream of gas comes from the crop storage chamber. A heat exchanger heats the stream of gas to thermally generate an aerosol of the stored crop treatment material. A blower moves the aerosol along the re-circulation loop to apply the aerosol to stored crops within the storage chamber. The circulation pathway includes a cooling gas loop comprising the crop storage chamber and a section of the re-circulation loop. Cooling gas from the crop storage chamber moves along the cooling gas loop into the section of the re-circulation loop to cool the heated stream of gas and aerosol prior to entering the crop storage chamber.

### BACKGROUND OF THE INVENTION

1. Technical Field

The invention relates generally to the application of treatments to stored crops. More particularly, the invention relates to the application of such treatments in aerosol form to stored crops within a crop storage facility. Specifically, the invention relates to the use of a heat exchanger to produce a thermally generated stored crop treatment aerosol which is circulated into and out of the crop storage facility with a substantially closed circulation pathway.

2. Background Information

The safe and effective storing of agronomic crops such as potato tubers has been a long standing need in the agricultural industry. In addition to providing tightly controlled conditions, such as ventilation, temperature, humidity and light, it is well known to treat stored crops with chemical compounds such as antimicrobials, fungicides, insecticides, growth regulators, disease controllers, combinations thereof and so forth. One growth regulator which has enjoyed long-standing use in the industry is known commonly as CIPC, also known as chlorpropham or isopropyl-N-3-chlorophenyl carbamate.

Aerosols of CIPC may be generated wherein CIPC is in a solid or liquid state for delivery into a crop storage facility in order to treat stored crops and in particular potato tubers. It has long been established that the size of the particles of CIPC is within the range of 1 to 10 microns and preferably from 1 to 5 microns in order to produce a suitable aerosol for delivery and treatment of the potatoes. While generation of aerosols at ambient or lower than ambient temperatures is known, the industry is nonetheless currently dominated by thermal aerosol generators wherein a stream of air is heated and/or the CIPC is heated so that the CIPC is vaporized in the heated stream of air to form the aerosol, which is delivered to the crop storage facility.

Several problems arise with regard to the use of thermal aerosol generators and with regard to the standard configuration of the crop storage facilities. One of the problems that arises in the current systems is the introduction of combustion products arising from the combustion apparatus which burns fuel in order to produce the heat needed to vaporize the CIPC or other stored crop treatment. The introduction of such combustion products and the thermal aerosol generation process raises at least two issues. First, the combustion products themselves are delivered along with the stored crop treatment aerosol into the crop storage facility so that the stored crops are thus exposed to these combustion products. Second, the heat that is used to vaporize the CIPC or other stored crop treatment may cause the chemical to react to produce a chemical derivative. Thus, the combustion products themselves and/or the chemical derivative of the stored crop treatment may create a compound which is supplied to the stored crops and which may not be safe or otherwise desirable, nor be within regulatory requirements such as EPA regulations. U.S. Pat. No. 5,723,184 granted to Yamamato and U.S. Pat. No. 6,068,888 granted to Forsythe et al. each teach the use of a heat exchanger for heating a stream of air to produce a stored crop treatment aerosol.

The heated air stream and stored crop treatment aerosol produced by thermal aerosol generators are typically introduced directly into the stored crop facility. Generally, direct introduction into the stored crop facility is not desirable because of the potential for a crop storage fire. Thus, it is desirable to cool the heated air stream and aerosol before it enters the crop storage facility. An improved configuration and method for cooling this heated aerosol is desirable within the art.

Another problem relates to the configuration of stored crop facilities, which are configured to receive a crop storage treatment through a duct of some sort and to exhaust air along with some of the suspended stored crop treatment therein out of the crop storage facility and into the environment. This configuration thus promotes environmental pollution and reduces the efficiency of the system by allowing the escape of a portion of the stored crop treatments which could otherwise be applied to the stored crops.

The present invention addresses these and other problems, as will be evident from the following description.

### BRIEF SUMMARY OF THE INVENTION

The present invention provides a method comprising the steps of heating with a heat exchanger a stream of gas at least a portion of which comes from a crop storage chamber to vaporize stored crop treatment material to thermally generate a stored crop treatment aerosol suspended in the heated stream of gas; and directing the stream of gas and aerosol into the crop storage chamber to treat crops therein with the aerosol.

The present invention also provides a stored crop treatment system comprising a crop storage chamber adapted to contain stored crops; a gas stream circulation pathway which includes a re-circulation loop comprising the crop storage chamber; a stored crop treatment feed mechanism in communication with the circulation pathway; the feed mechanism being adapted to feed stored crop treatment material into the circulation pathway; and a heat exchanger for transferring heat into the circulation pathway whereby the heat exchanger is adapted to heat a gas stream including gas from the crop storage chamber to produce a thermally generated aerosol of the stored crop treatment material for application to the stored crops within the storage chamber.

### BRIEF DESCRIPTION OF THE SEVERAL VIEWS OF THE DRAWINGS

FIG. 1 is a diagrammatic view of a first embodiment of the stored crop treatment aerosol generation and delivery system of the present invention.

FIG. 2 is a diagrammatic view of a second embodiment of the stored crop treatment aerosol generation and delivery system of the present invention.

Similar numbers refer to similar parts throughout the specification.

### DETAILED DESCRIPTION OF THE INVENTION

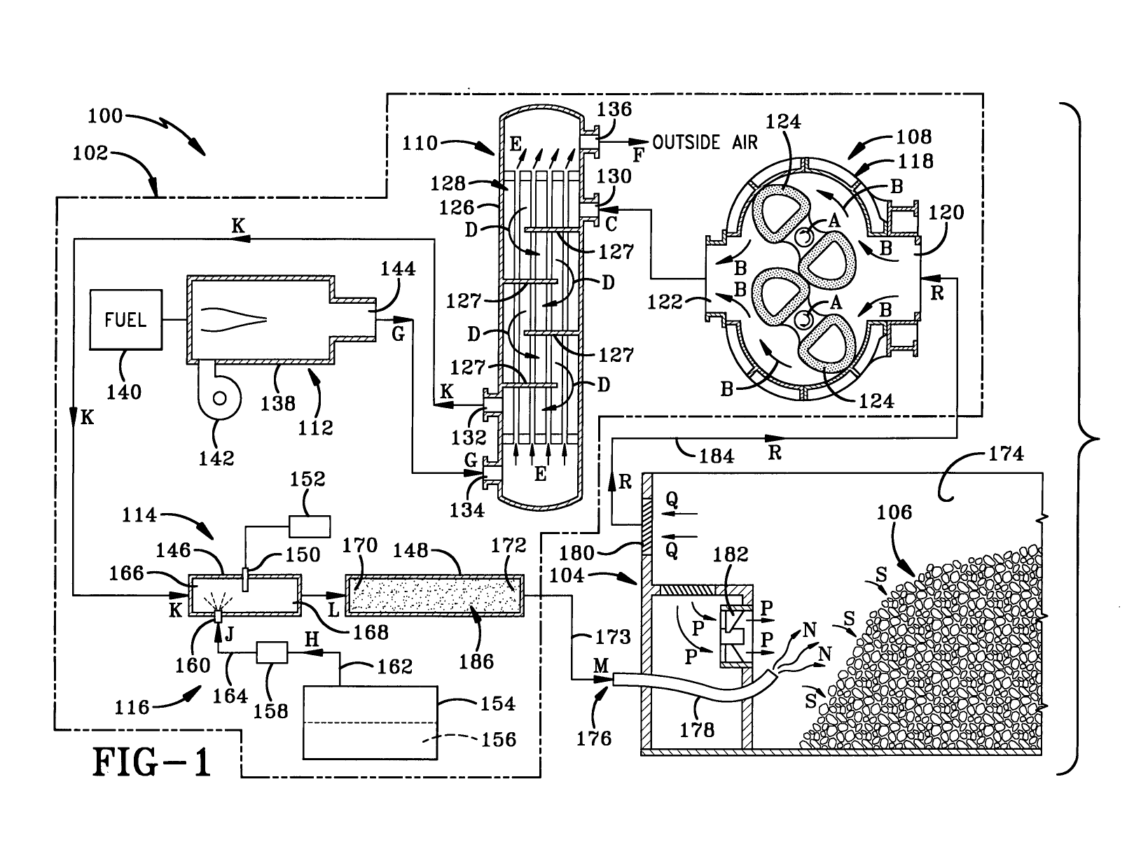

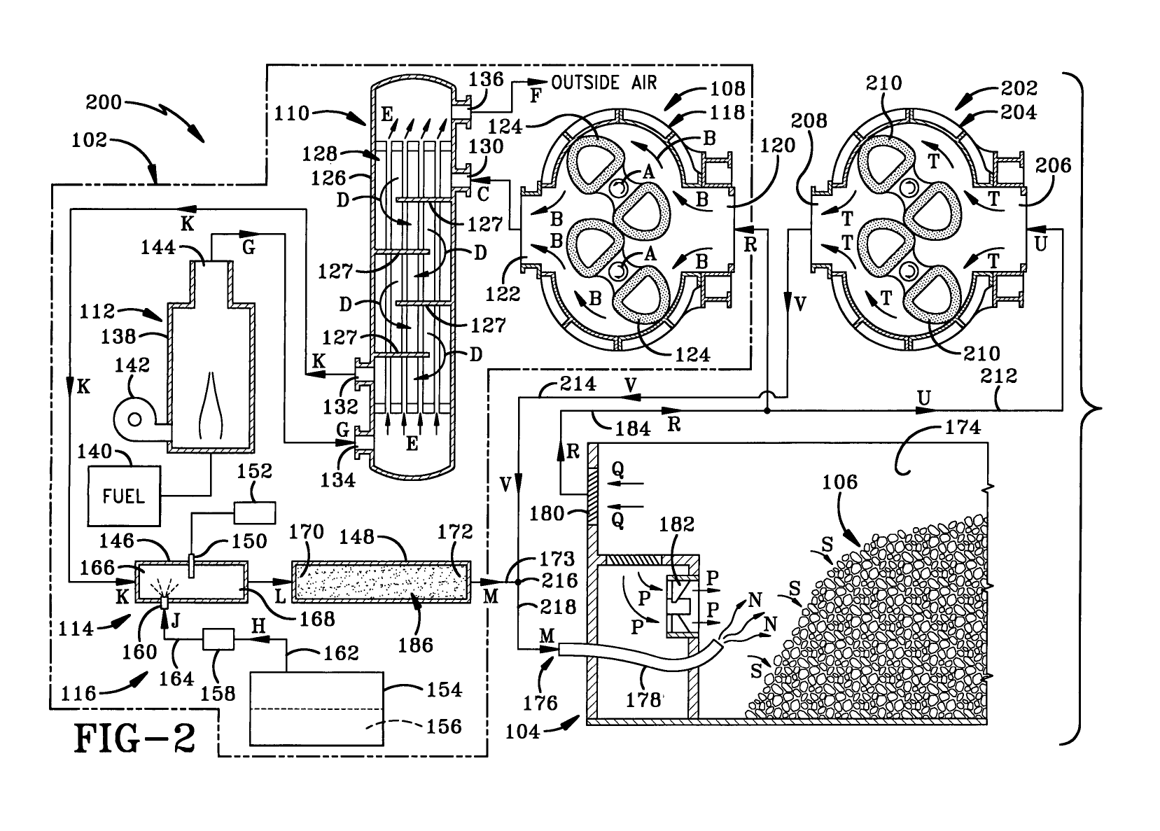

A first embodiment of the stored crop treatment aerosol generation and delivery system of the present invention is indicated generally at 100 in FIG. 1; and a second embodiment of the system is indicated generally at 200 in FIG. 2. In general, system 100 is configured to generate a stored crop treatment aerosol and circulate the aerosol into and out of a crop storage facility whereby storage air from the crop facility and aerosol which is passed through the crop storage facility is recycled within system 100. More particularly, system 100 includes a thermal aerosol generator 102 and a crop storage facility 104 for storing crops 106 such as potatoes.

Thermal aerosol generator 102 includes a re-circulation blower 108 in the form of a gas pump or air pump, a heat exchanger 110, a heat source 112 for providing heat to heat exchanger 110, an aerosol production zone 114 and a stored crop treatment feed mechanism 116. While any blower 108 which is suitable to the purpose may be used, blower 108 is shown as a two-impeller type of rotary positive-displacement blower. Blower 108 includes a housing 118 defining an inlet 120 and an outlet 122. Blower 108 further includes a pair of impellers 124 which rotate in opposite directions as indicated by Arrows A in order to move a gas such as air into inlet 120 and out of outlet 122 as indicated by Arrows B.

Heat exchanger 110 may be any type of heat exchanger suitable for the purpose. One example is an electrically operated heat exchanger although other heat exchangers may be used which are capable of producing sufficient heat to produce a stored crop treatment aerosol. Heat exchanger 110 is shown more particularly as a shell-and-tube heat exchanger with a one-shell pass and a one-tube pass. Heat exchanger 110 is disposed downstream from blower 108 as indicated by Arrow C. Heat exchanger 110 includes a shell 126 and a tube or tubes 128 disposed within shell 126. Heat exchanger 110 includes a plurality of baffles 127 extending inwardly from shell 126. Heat exchanger 110 further includes a gas stream inlet 130 and a gas stream outlet 132 whereby a gas stream may move into inlet 130, along tubes 128 and around baffles 127, as indicated by Arrows D, and out of outlet 132. Gas stream inlet 130 is in fluid communication with outlet 122 of blower 108, as indicated by Arrow C which represents an air stream. Gas stream inlet 130 may be connected directly to outlet 122 or alternately may be connected by a duct, which may also be represented by Arrow C. Heat exchanger 110 further includes an exhaust stream inlet 134 and an exhaust stream outlet 136 each of which is in fluid communication with tube or tubes 128 so that exhaust fumes may travel into inlet 134, through tubes 128 as indicated at Arrows E and out of outlet 136 to outside air as indicated at Arrow F.

Heat source 112 includes a combustion chamber 138, a source of fuel 140 for supplying fuel into combustion chamber 138 and a blower 140 for blowing air or another source of oxygen into combustion chamber 138. Combustion chamber 138 includes an exhaust outlet 144 which is in fluid communication with exhaust stream inlet 134 of heat exchanger 110 whereby exhaust fumes or an exhaust stream may flow out of exhaust outlet 144 into exhaust stream inlet 134. Exhaust outlet 144 may be connected directly to exhaust stream inlet 134 or may be connected by a duct, which may be represented by Arrows G.

Within aerosol production zone 114 are situated a feed passage 146 and a vaporization area or passage 148. A thermocouple 150 extends into feed passage 146 and is in electrical communication with a temperature readout 152.

Feed mechanism 116 includes a stored crop treatment container 154 for containing stored crop treatment material 156 therein, a metered pump 158 and a nozzle 160. A supply tube 162 extends between and is connected to container 154 and pump 158 whereby container 154 and pump 158 are in fluid communication with one another. A feed tube 164 extends between and is connected to pump 158 and nozzle 160 whereby pump 158 and nozzle 160 are in fluid communication with one another. Feed mechanism 116 is thus configured so that pump 158 is able to pump stored crop treatment material 156 from container 154 via supply tube 162 into pump 158 as indicated by Arrow H and from pump 158 to nozzle 160 via feed tube 164 as indicated by Arrow J and into feed passage 146, as indicated by the dashed lines therein. Feed mechanism 116 is configured particularly for feeding stored crop treatment material 156 in liquid form, whether this be a liquid at ambient temperature, a material which has been heated to form a molten material or utilized as a solvent in which material 156 is dissolved. However, feed mechanism 116 also represents a mechanism for feeding stored crop treatment material 156 in solid form.

Feed passage 146 has an inlet 166 and an outlet 168 whereby inlet 166 is in fluid communication with gas stream outlet 132 of heat exchanger 110, as indicated by Arrows K. Inlet 166 may be connected to gas stream outlet 132 or they may be joined by a duct, which may be represented by Arrows K. Vaporization passage 148 have an inlet 170 and an outlet 172 with inlet 170 being in fluid communication with outlet 168 of feed passage 146, as indicated by Arrow L. Typically, feed passage 146 and vaporization passage 148 may be thought of as a single continuous passage. Thus, a gas stream may move as indicated at Arrows K into feed passage 146 and then into vaporization passage 148 as indicated by Arrow L. An aerosol generator exhaust duct or passage 173 extends downstream from outlet 172.

Crop storage facility 104 defines a crop storage chamber 174 and has an entrance opening 176 defined by an entrance duct 178. Exhaust passage 173 extends immediately upstream from entrance opening 176. Entrance opening 176 is thus in fluid communication with exhaust passage 173 and crop storage chamber 174 to allow a gas stream to move from passage 173 into entrance opening 176 as indicated at Arrow M and into chamber 174 via duct 178 as indicated by Arrows N. Storage chamber 174 also has a vent or exit opening 180 which is in fluid communication with crop storage chamber 174. Crop storage facility 104 also includes an internal circulation blower 182 for moving air or another gas within storage chamber 174 as indicated by Arrows P. A duct 184 extends between and is mounted on crop storage facility 104 and blower 108. Exit opening 180 of facility 104 is in fluid communication with inlet 120 of blower 108 via duct 184 so that a gas stream or air stream may move out of storage chamber 174 via exit opening 180, as indicated by Arrows Q, into duct 184 and through duct 184 as indicated by Arrows R into inlet 120 of blower 108. Thus, system 100 defines a gas stream circulation pathway which includes a closed or substantially closed re-circulation loop or gas passage, as indicated generally by Arrows B, C, D, K, L, M, N, Q and R. Thus, as detailed below, the re-circulation loop is capable of circulating air or another gas and any stored crop treatment aerosol from crop storage chamber 174 and through thermal aerosol generator 102 and then back into storage chamber 174. More particularly, this includes moving said air and aerosol from chamber 174 through blower 108, heat exchanger 110, feed passage 146, vaporization passage 148 and back into storage chamber 174 in a cyclical manner.

In operation, system 100 functions as follows. Blower 108 is configured to circulate or cycle a gas stream or air stream along the re-circulation loop noted above. Blower 108 is operated to rotate impellers 124 in order to move a gas stream or air stream which includes air or gas from within crop storage chamber 174 into inlet 120 of blower 108, as indicated by Arrows Q, R and B. The gas stream then moves as indicated by Arrows B and C from outlet 122 of blower 108 into gas stream inlet 130 of heat exchanger 110. The gas stream then moves through heat exchanger 110 as indicated by Arrows D and out of heat exchanger 110 via gas stream outlet 132. Meanwhile, heat source 112 is operated to produce heated combustion fumes which heat exchanger 110. Heat exchanger 110 then transfers heat to a portion of the re-circulation loop disposed within heat exchanger 110, thus transferring heat to the gas stream passing therethrough as indicated by Arrows D.

More particularly, fuel is pumped from source of fuel 140 into combustion chamber 138 as blower 142 blows air or another source of oxygen into combustion chamber 138 so that the fuel is burned within combustion chamber 138 to reduce heat and exhaust fumes or an exhaust stream which exits fuel chamber 138 and travels as indicated by Arrows G into heat exchanger 110 via exhaust stream inlet 134. The exhaust stream then travels through tubes 128 as indicated by Arrows E and out of exhaust stream outlet 136 as indicated by Arrow F into the outside air so that combustion gases or fumes do not enter the gas stream which is circulating within the circulation pathway of system 100. The exhaust stream then heats the gas stream within the circulation pathway as the gas stream moves through heat exchanger 110 along the path indicated at Arrows D and the exhaust stream travels through heat exchanger 110 as indicated at Arrows E.

Heat exchanger 110 thus heats the gas stream to a vaporization-inducing temperature to vaporize the stored crop treatment 156 within feed passage 146 and vaporization passage 148. More particularly, the gas stream travels from heat exchanger 110 as indicated by Arrows K into feed passage 146. In general, the vaporization-inducing temperature produced by heat exchanger 110 within feed passage 146 will be greater than or equal to the vaporization temperature of stored crop treatment material 156 and thus heat exchanger 110 is typically heated to a higher temperature than the vaporization-inducing temperature in order to produce sufficient heat within the gas stream. While the present invention is useful for various stored crop treatment materials 156, it is noted that CIPC has a melting temperature of approximately 105° F. and a vaporization temperature of about 475° F. Thus, where CIPC is in a solvent form, the vaporization temperature may vary as a result. Typically, CIPC may be thermally generated to create an aerosol at or above about 500° F. and may be thermally generated using an airstream temperature up to about 1100° F. without or without substantial degradation of CIPC as long as the residence time within these high range temperatures is sufficiently short. Many thermal aerosol generators operate to give a vaporized CIPC airstream within a range of about 700° F. to 850° F., typically about 800° F.

Meanwhile, pump 158 pumps stored crop treatment material 156 via supply tube 162, feed tube 164 and nozzle 160 into feed passage 146 where the heated gas stream heats the material 156 to cause vaporization thereof within vaporization passage 148. As previously noted, feeding mechanism 116 may feed material 156 in either liquid form or in solid form into feed passage 146. Thermocouple 150 and temperature readout 152 are provided in order to monitor and control the temperature within feed passage 146. Vaporization of material 156 thus creates a stored crop treatment aerosol 186 of material 156 which travels from vaporization passage 148 into crop storage chamber 174 via passage 173 and entrance opening 176 as indicated by Arrows M and N. Stored crop treatment aerosol 186 then moves into the spaces between the stored crops 106 as indicated by Arrows S.

The storage gas or storage air within chamber 174 then moves along with a portion of the stored crop treatment aerosol out of exit opening 180 as indicated by Arrows Q and into blower 108 via duct 184 and inlet 120 of blower 108 as indicated by Arrows R and B. The flow of the gas stream or air stream with stored crop treatment aerosol 186 suspended therein is then circulated or cycled through the re-circulation loop as previously described with regard to the gas stream alone so that the gas stream and aerosol therein coming from crop storage chamber 174 are heated by heat exchanger 110 and then moved into feed passage 146 where additional stored crop treatment aerosol 186 is produced as previously described and the portion of aerosol 186 which traveled along the re-circulation loop from storage chamber 174 is added to the additional aerosol and cycled back into storage chamber 174 via passage 173 and entrance opening 176.

The aerosol 186 which is moved from aerosol generator 102 into crop storage chamber 174 and from chamber 174 back through heat exchanger 110 and into feed passage 146 are predominantly in the range of 1 to 10 microns in diameter, as particles which are above 10 microns will tend to fall out by gravity within the gas stream. More preferably, material 156 will have been condensed and agglomerated or coagulated to sizes ranging from 1 to 5 microns.

Thus, system 100 allows for the use of storage gas or storage air coming from storage chamber 174 in the production of stored crop treatment aerosol 186, which is then moved back into chamber 174 in order to treat crops 106 therewith. System 100 also allows the use of any portion of aerosol 186 that has entered chamber 174 but which has not been deposited on crops 106 or on any of the structure of system 100 to be recirculated via blower 108, heated by heat exchanger 110 and mixed with additional aerosol 186 being produced in feed passage 146 and vaporization passage 148. This mixture of the portion of aerosol 186 from chamber 174 and the additional aerosol 186 creates an increased concentration of aerosol 186 which moves to chamber 174 via passage 173 to treat crops 106. Thus, the portion of aerosol 186 which came from storage chamber 174 is recirculated or recycled back to storage chamber 174. As previously noted, this recirculation process prevents the displacement of aerosol 186 into the environment external to the storage crop facility 104, thus preventing environmental pollution and making system 100 more efficient by allowing the reuse of the recirculated aerosol. In addition, in contrast to known prior art, where system 100 has a closed circulation pathway, it eliminates the need for a filter to filter outside air used in generating a stored crop treatment aerosol, because no outside air is used and use of a filter would reduce the amount of effective aerosol which is recycled.

In addition, system 100 provides for the thermal generation of a stored crop treatment aerosol wherein heat exchanger 110 provides the heat necessary to vaporize the stored crop treatment 156 to produce the aerosol. As previously noted, this prevents the introduction of combustion gasses or fumes into the gas stream circulation pathway, as discussed in the Background section of the present application. Thus, combustion gasses do not enter crop storage chamber 174 so that no combustion products may be deposited on stored crops 106.

System 200 is similar to system 100 except that it includes a cooling assembly for cooling the heated gas stream and aerosol being produced by thermal aerosol generator 102 before entering crop storage facility 104. More particularly, this cooling system includes a cooling gas blower 202 for circulating relatively cool air or other gas from storage chamber 174 to mix with the heated gas stream and aerosol coming from thermal aerosol generator 102 in order to cool the same prior to entering storage chamber 174. Cooling blower 202 has the same configuration as blower 108 and includes a housing 204 defining an inlet 206 and an outlet 208. Blower 202 includes a pair of impellers 210 which operate in the same fashion as that of blower 108 in order to move a gas stream into inlet 206 and out of outlet 208 as indicated by Arrows T.

More particularly, cooling blower 202 is downstream of crop storage chamber 174 with inlet 206 in fluid communication with exit opening 180 via duct 184 as indicated by Arrows R and U. More particularly, a duct or passage 212 extends between and is in fluid communication with passage 184 and inlet 206. The cooling system further includes another duct or passage 214 which is in fluid communication with outlet 208 of blower 202 and entrance opening 176 of crop storage facility 104 via passage 173 at an intersection 216 of passage 173 and passage 214. More particularly, passage 173 includes a cooling section 218 which extends downstream from intersection 216 to entrance opening 176. Thus, section 218 extends from and is immediately upstream of entrance opening 176. Thus, the gas stream circulation pathway of system 200 includes the recirculation pathway of system 100 and adds a cooling gas loop or passage which includes crop storage chamber 174, a section of passage 184, passage 212, blower 202, passage 214 and section 218 of passage 173. The cooling gas loop thus follows the basic path indicated sequentially at Arrows Q, R, U, T, V, M and N. It is further noted that as shown in FIG. 2, passage 212 is coincident with a portion of passage 184 immediately downstream of entrance opening 180 of crop storage facility 104. However, passage 212 need not overlap with passage 184 and indeed may come from a separate exit opening of facility 104 if desired.

In operation, system 200 functions like system 100 except that system 200 cools the heated gas stream and aerosol 186 within section 218 of passage 173. More particularly, blower 202 is operated to move cooling gas or air from crop storage chamber 174 as indicated by Arrows Q into passages 184 and 212 and into blower 202 via inlet 206, as indicated at Arrows T. This cooling gas stream then is pumped via outlet 208 into passage 214 as indicated at Arrows V and into section 218 of passage 173 at intersection 216 to mix with and thereby cool the heated air stream and aerosol prior to entering crop storage chamber 174 via entrance opening 176 as indicated at Arrows M and N. It is noted that the various inlets within systems 100 and 200 as well as the entrance opening to the storage facility are all gas stream-receiving and that the various outlets and the exit opening of the storage facility are all gas stream-exhausting.

Thus, system 200 cools the heated gas stream and aerosol formed by thermal aerosol generator 102 prior to entry into crop storage chamber 174 in order to minimize the potential for a crop storage fire upon entry into chamber 174. In addition, system 200 uses storage air or storage gas within storage chamber 174 to provide the cooling gas, thus preventing displacement of the aerosol into the environment. Thus, the circulation pathway of system 200 is a closed or substantially closed gas passage system. System 200 also eliminates or minimizes the need for a separate cooler in order to cool the heated gas stream and aerosol prior to entering the crop storage facility. This is possible because crop storage facilities are typically a very large heat sink with respect to the amount of heat that is introduced. Alternately, a separate cooler may be used if needed or desired.

As previously noted, any thermal aerosol generator may be used with the invention. For example, one thermal aerosol generator, which is known as the LECO machine, is manufactured by Lowndes Engineering Company of Valdosta, Ga. Another such generator is known as the TIFA machine manufactured by Todd Shipyard Corp. of Houston, Tex. Each of these machines may be modified by replacing the combustion system normally used therein with the heat exchanger of the present invention.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of the invention is an example and the invention is not limited to the exact details shown or described.

1. A method comprising the steps of:
heating with a heat exchanger a stream of gas at least a portion of which comes from a crop storage chamber to vaporize stored crop treatment material to thermally generate a stored crop treatment aerosol suspended in the heated stream of gas; and
directing the stream of gas and aerosol into the crop storage chamber to treat crops therein with the aerosol.

2. The method of claim 1 further including the step of cooling the heated stream of gas and aerosol prior to entry thereof into the crop storage chamber.

3. The method of claim 2 wherein the step of cooling includes the step of introducing cooling gas into the stream of gas.

4. The method of claim 3 wherein the step of introducing includes the step of introducing cooling gas from the crop storage chamber into the stream of gas.

5. The method of claim 3 wherein the step of introducing includes the step of introducing cooling gas into the heated stream of gas wherein substantially all of the cooling gas comes from the crop storage chamber.

6. The method of claim 5 wherein the step of cooling includes the step of introducing cooling gas into the heated stream of gas within a cooling passage which extends upstream from and is in fluid communication with an entrance opening of the crop storage chamber.

7. The method of claim 1 wherein the step of heating includes the step of heating a stream of gas substantially all of which comes from the crop storage chamber.

8. The method of claim 7 further including the step of cooling the heated stream of gas and aerosol prior to entry thereof into the crop storage chamber.

9. The method of claim 8 wherein the step of cooling includes the step of introducing cooling gas from the crop storage chamber into the heated stream of gas.

10. The method of claim 8 wherein the step of cooling includes the step of introducing cooling gas into the heated stream of gas wherein substantially all of the cooling gas comes from the crop storage chamber.

11. The method of claim 10 wherein the step of heating includes the step of heating with a heat exchanger a stream of gas in which is suspended a stored crop treatment aerosol wherein substantially all of the gas and the aerosol come from the crop storage chamber.

12. The method of claim 7 wherein the step of heating includes the step of heating with the heat exchanger a stream of gas in which is suspended a stored crop treatment aerosol wherein substantially all of the gas and the aerosol come from the crop storage chamber.

13. The method of claim 1 further including the steps of heating with the heat exchanger a stream of gas in which is suspended a portion of the thermally generated stored crop treatment aerosol wherein the portion of the aerosol comes from the crop storage chamber and wherein additional stored crop treatment aerosol is thermally generated; and directing the portion of the aerosol and the additional aerosol into the crop storage chamber.

14. A stored crop treatment system comprising:
a crop storage chamber adapted to contain stored crops;
a gas stream circulation pathway which includes a re-circulation loop comprising the crop storage chamber;
a stored crop treatment feed mechanism in communication with the circulation pathway; the feed mechanism being adapted to feed stored crop treatment material into the circulation pathway; and
a heat exchanger for transferring heat into the circulation pathway whereby the heat exchanger is adapted to heat a gas stream including gas from the crop storage chamber to produce a thermally generated aerosol of the stored crop treatment material for application to the stored crops within the storage chamber.

15. The system of claim 14 wherein the gas stream circulation pathway is a substantially closed gas passage system whereby the heat exchanger is adapted to heat the gas stream substantially all of which comes from the crop storage chamber.

16. The system of claim 14 wherein the gas stream circulation pathway includes a cooling gas loop which comprises the crop storage chamber and a section of the re-circulation loop which extends upstream from the crop storage chamber whereby the cooling gas loop is adapted to introduce cooling gas from the crop storage chamber into the section of the re-circulation loop.

17. The system of claim 16 wherein a cooling gas blower is in fluid communication with the cooling loop whereby the blower is adapted to move cooling gas from the crop storage chamber into the section of the re-circulation loop.

18. The system of claim 17 wherein the heat exchanger is part of a thermal aerosol generator having an aerosol outlet; wherein the re-circulation loop includes an aerosol generator exhaust passage connected to and in fluid communication with the aerosol outlet; and wherein the aerosol generator exhaust passage includes the section of the re-circulation loop which extends upstream from the crop storage chamber.

19. The system of claim 16 wherein the gas stream circulation pathway is a substantially closed gas passage system whereby the cooling gas loop is adapted to introduce the cooling gas substantially all of which comes from the crop storage chamber and whereby the heat exchanger is adapted to heat the gas stream substantially all of which comes from the crop storage chamber.

20. The system of claim 14 wherein the crop storage chamber has a gas stream-receiving entrance opening; wherein the gas stream circulation pathway includes a-cooling gas passage which intersects the re-circulation loop at an intersection; wherein the re-circulation loop includes a cooling section which extends downstream from the intersection to the entrance opening of the crop storage chamber whereby the cooling gas passage is adapted to introduce cooling gas into the cooling section of the re-circulation loop.
